SEQUENCE LISTING (128 sequences)

SEQ ID NO: 1 - protein, 8 aa, Homo sapiens
  MISC_FEATURE (4)..(4): Hydroxyproline
EGPPGPQG

SEQ ID NO: 2 - protein, 7 aa, Homo sapiens
  MISC_FEATURE (3)..(3): Hydroxyproline
GPPGPQG

SEQ ID NO: 3 - protein, 7 aa, Homo sapiens
GPPGPQG

SEQ ID NO: 4 - protein, 9 aa, Homo sapiens
GEPGDDAPS

SEQ ID NO: 5 - protein, 20 aa, Bos taurus
PGDDGEAGKPGKSGERGPPG

SEQ ID NO: 6 - protein, 20 aa, Homo sapiens
PGDDGEAGKPGKAGERGPPG

SEQ ID NO: 7 - protein, 7 aa, Homo sapiens
GPPGPQG

SEQ ID NO: 8 - protein, 10 aa, Homo sapiens
GEPGDDGPSG

SEQ ID NO: 9 - protein, 13 aa, Homo sapiens
APGEDGRPGPPGP

SEQ ID NO: 10 - protein, 16 aa, Homo sapiens
GKVGPSGAPGEDGRPG

SEQ ID NO: 11 - protein, 9 aa, Homo sapiens
AEGPPGPQG

SEQ ID NO: 12 - protein, 10 aa, Homo sapiens
GPPGPQGLAG

SEQ ID NO: 13 - protein, 9 aa, Homo sapiens
GEPGDDGPS

SEQ ID NO: 14 - protein, 16 aa, Homo sapiens
GEPGDDGPSGAEGPPG

SEQ ID NO: 15 - protein, 21 aa, Homo sapiens
EKGEPGDDAPSGAEGPPGPQG

SEQ ID NO: 16 - protein, 21 aa, Homo sapiens
GPPGPPGKPGDDGEAGKPGKA

SEQ ID NO: 17 - protein, 21 aa, Homo sapiens
GPPGPRGRSGETGPAGPPGNP

SEQ ID NO: 18 - protein, 22 aa, Homo sapiens
GAPGPQGFQGNPGEPGEPGVSY

SEQ ID NO: 19 - protein, 20 aa, Homo sapiens
GEPGDDAGPSGAEGPPGPQG

SEQ ID NO: 20 - protein, 7 aa, Homo sapiens
GEAGKPG

SEQ ID NO: 21 - protein, 23 aa, Homo sapiens
CGKVGPSGAPGEDGRPGPPGPQY

SEQ ID NO: 22 - protein, 13 aa, Homo sapiens
APGEDGRPGPPGP

SEQ ID NO: 23 - protein, 9 aa, Homo sapiens
HRGYPGLDG

SEQ ID NO: 24 - protein, 4 aa, Homo sapiens
GQPG

SEQ ID NO: 25 - protein, 11 aa, Homo sapiens
CGGEGPPGPQG

SEQ ID NO: 26 - protein, 19 aa, Homo sapiens
GAEGPPGPQGLAGQRGIVG

SEQ ID NO: 27 - protein, 19 aa, Homo sapiens
GAPGTPGPQGIAGQRGVVG

SEQ ID NO: 28 - protein, 19 aa, Homo sapiens
GPPGTPGPQGLLGAPGILG

SEQ ID NO: 29 - protein, 19 aa, Homo sapiens
GPPGAPGPLGIAGITGARG

SEQ ID NO: 30 - protein, 12 aa, Homo sapiens
CGGEGPPGPQGL

SEQ ID NO: 31 - protein, 13 aa, Homo sapiens
CGGEGPPGPQGLA

SEQ ID NO: 32 - protein, 10 aa, Homo sapiens
CGGEGPPGPQ

SEQ ID NO: 33 - protein, 9 aa, Homo sapiens
CGGEGPPGP

SEQ ID NO: 34 - protein, 8 aa, Homo sapiens
CGPPGPQG

SEQ ID NO: 35 - protein, 30 aa, Homo sapiens
LQGPAGPPGEKGEPGDDGPSGAEGPPGPQG

SEQ ID NO: 36 - protein, 5 aa, Homo sapiens
PGPQG

SEQ ID NO: 37 - protein, 6 aa, Homo sapiens
PPGPQG

SEQ ID NO: 38 - protein, 31 aa, Homo sapiens
VLQGPAGPPGEKGEPGDDGPSGAEGPPGPQG

SEQ ID NO: 39 - protein, 47 aa, Homo sapiens
KGARGDSGPPGRAGEPGLQGPAGPPGEKGEPGDDGPSGAEGPPGPQG

SEQ ID NO: 40 - protein, 45 aa, Homo sapiens
ARGDSGPPGRAGEPGLQGPAGPPGEKGEPGDDGPSGAEGPPGPQG

SEQ ID NO: 41 - protein, 28 aa, Homo sapiens
GPAGPPGEKGEPGDDGPSGAEGPPGPQG

SEQ ID NO: 42 - protein, 145 aa, Homo sapiens
GPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLGLPGKDGETGAEGPPPA

SEQ ID NO: 43 - protein, 347 aa, Homo sapiens
MPGERGAAGIAGPKGDRGDVGEKGPEGAPGKDGGRGLTGPIGPPGPAGANGEKGEVGPPGPAGSAGARGAPGERGETGPPGTSGIAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGQQGAPGASGDRGPPGPVGPPGLTGPAGEPGREGSPGADGPPGRDGAAGVKGDRGETGAVGAPGAPGPPGSPGPAGPTGKQGDRGEAGAQGPM

SEQ ID NO: 44 - protein, 6 aa, Homo sapiens
EKGPDP

SEQ ID NO: 45 - protein, 7 aa, Homo sapiens
  MISC_FEATURE (4)..(5): Isomerised peptide bond
GDIKDIV

SEQ ID NO: 46 - protein, 5 aa, Homo sapiens
  MISC_FEATURE (4)..(5): Isomerised peptide bond
EKGPD

SEQ ID NO: 47 - protein, 7 aa, Homo sapiens
GDIKDIV

SEQ ID NO: 48 - protein, 5 aa, Homo sapiens
EKGPD

SEQ ID NO: 49 - protein, 5 aa, Homo sapiens
PGVKG

SEQ ID NO: 50 - protein, 6 aa, Homo sapiens
PGPKGE

SEQ ID NO: 51 - protein, 6 aa, Homo sapiens
GQKGEP

SEQ ID NO: 52 - protein, 13 aa, Homo sapiens
  MISC_FEATURE (6)..(6): Undefined amino acid
GFQGLXGXXGXXG

SEQ ID NO: 53 - protein, 13 aa, Homo sapiens
  MISC_FEATURE (6)..(6): undefined amino acid
GLQGLXGXXGXSG

SEQ ID NO: 54 - protein, 6 aa, Homo sapiens
DQGVPG

SEQ ID NO: 55 - protein, 6 aa, Homo sapiens
REGSPG

SEQ ID NO: 56 - protein, 6 aa, Homo sapiens
LAGPKG

SEQ ID NO: 57 - protein, 6 aa, Homo sapiens
LTGPAG

SEQ ID NO: 58 - protein, 6 aa, Homo sapiens
PKGARG

SEQ ID NO: 59 - protein, 6 aa, Homo sapiens
GQPGPA

SEQ ID NO: 60 - protein, 6 aa, Homo sapiens
EPGGVG

SEQ ID NO: 61 - protein, 6 aa, Homo sapiens
RDGAAG

SEQ ID NO: 62 - protein, 27 aa, Homo sapiens
LTGPAGEPGREGSPGADGPPGRDGAAG

SEQ ID NO: 63 - protein, 18 aa, Homo sapiens
REGSPGADGPPGRDGAAG

SEQ ID NO: 64 - protein, 16 aa, Homo sapiens
AQGPPGATGFPGAAGR

SEQ ID NO: 65 - protein, 11 aa, Homo sapiens
ASGDRGPPGPV

SEQ ID NO: 66 - protein, 15 aa, Homo sapiens
ASGDRGPPGPVGPPG

SEQ ID NO: 67 - protein, 15 aa, Homo sapiens
GANGEKGEVGPPGPA

SEQ ID NO: 68 - protein, 15 aa, Homo sapiens
GAPGEDGRPGPPGPQ

SEQ ID NO: 69 - protein, 18 aa, Homo sapiens
GARGAPGERGETGPPGPA

SEQ ID NO: 70 - protein, 9 aa, Homo sapiens
GDRGPPGPV

SEQ ID NO: 71 - protein, 7 aa, Homo sapiens
GERGFPG

SEQ ID NO: 72 - protein, 9 aa, Homo sapiens
GERGFPGER

SEQ ID NO: 73 - protein, 15 aa, Homo sapiens
GESGSPGENGSPGPM

SEQ ID NO: 74 - protein, 16 aa, Homo sapiens
GLPGPPGPPGEGGKPG

SEQ ID NO: 75 - protein, 9 aa, Homo sapiens
GPIGPPGPA

SEQ ID NO: 76 - protein, 19 aa, Homo sapiens
GPPGPPGKPGDDGEAGKPG

SEQ ID NO: 77 - protein, 6 aa, Homo sapiens
GPPGPV

SEQ ID NO: 78 - protein, 9 aa, Homo sapiens
GPPGPVGPA

SEQ ID NO: 79 - protein, 15 aa, Homo sapiens
LPGPPGPPGEGGKPG

SEQ ID NO: 80 - protein, 14 aa, Homo sapiens
NPGPPGPPGPPGPG

SEQ ID NO: 81 - protein, 5 aa, Homo sapiens
PIGPP

SEQ ID NO: 82 - protein, 20 aa, Homo sapiens
REGSPGADGPPGRDGAAGVK

SEQ ID NO: 83 - protein, 14 aa, Homo sapiens
SNGNPGPPGPPGPS

SEQ ID NO: 84 - protein, 1060 aa, Homo sapiens
QMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRA

SEQ ID NO: 85 - protein, 23 aa, Homo sapiens
AAGARGNDGQPGPAGPPGPVGPA

SEQ ID NO: 86 - protein, 18 aa, Homo sapiens
AQGPRGEPGTPGSPGPAG

SEQ ID NO: 87 - protein, 18 aa, Homo sapiens
ARGAPGERGETGPPGPAG

SEQ ID NO: 88 - protein, 11 aa, Homo sapiens
ASGDRGPPGPV

SEQ ID NO: 89 - protein, 12 aa, Homo sapiens
ASGDRGPPGPVG

SEQ ID NO: 90 - protein, 9 aa, Homo sapiens
ATGPLGPKG

SEQ ID NO: 91 - protein, 12 aa, Homo sapiens
DRGPPGPVGPPG

SEQ ID NO: 92 - protein, 30 aa, Homo sapiens
ERGAPGNRGFPGQDGLAGPKGAPGERGPSG

SEQ ID NO: 93 - protein, 35 aa, Homo sapiens
FQGLPGPPGPPGEGGKPGDQGVPGEAGAPGLVGPR

SEQ ID NO: 94 - protein, 36 aa, Homo sapiens
FQGLPGPPGPPGEGGKPGDQGVPGEAGAPGLVGPRG

SEQ ID NO: 95 - protein, 15 aa, Homo sapiens
FTGLQGLPGPPGPSG

SEQ ID NO: 96 - protein, 39 aa, Homo sapiens
FTGLQGLPGPPGPSGDQGASGPAGPSGPRGPPGPVGPSG

SEQ ID NO: 97 - protein, 15 aa, Homo sapiens
GANGEKGEVGPPGPA

SEQ ID NO: 98 - protein, 15 aa, Homo sapiens
GAPGEDGRPGPPGPQ

SEQ ID NO: 99 - protein, 16 aa, Homo sapiens
GAPGEDGRPGPPGPQG

SEQ ID NO: 100 - protein, 15 aa, Homo sapiens
GAPGERGETGPPGPA

SEQ ID NO: 101 - protein, 15 aa, Homo sapiens
GESGSPGENGSPGPM

SEQ ID NO: 102 - protein, 9 aa, Homo sapiens
GPIGPPGPA

SEQ ID NO: 103 - protein, 6 aa, Homo sapiens
GPPGPV

SEQ ID NO: 104 - protein, 10 aa, Homo sapiens
GPPGPVGPPG

SEQ ID NO: 105 - protein, 16 aa, Homo sapiens
GPRGPPGPAGAPGPQG

SEQ ID NO: 106 - protein, 26 aa, Homo sapiens
GVMQGPMGPMGPRGPPGPAGAPGPQG

SEQ ID NO: 107 - protein, 25 aa, Homo sapiens
IVGLPGQRGERGFPGLPGPSGEPGK

SEQ ID NO: 108 - protein, 21 aa, Homo sapiens
KQGDRGEAGAQGPMGPSGPAG

SEQ ID NO: 109 - protein, 21 aa, Homo sapiens
KVGPSGAPGEDGRPGPPGPQG

SEQ ID NO: 110 - protein, 21 aa, Homo sapiens
LPGKDGETGAAGPPGPAGPAG

SEQ ID NO: 111 - protein, 33 aa, Homo sapiens
LPGKDGETGAAGPPGPAGPAGERGEQGAPGPSG

SEQ ID NO: 112 - protein, 36 aa, Homo sapiens
LQGLPGPPGPSGDQGASGPAGPSGPRGPPGPVGPSG

SEQ ID NO: 113 - protein, 17 aa, Homo sapiens
LTGPAGEPGREGSPGAD

SEQ ID NO: 114 - protein, 10 aa, Homo sapiens
GPPGRDGAAG

SEQ ID NO: 115 - protein, 12 aa, Homo sapiens
LTGPIGPPGPAG

SEQ ID NO: 116 - protein, 33 aa, Homo sapiens
LTGRPGDAGPQGKVGPSGAPGEDGRPGPPGPQG

SEQ ID NO: 117 - protein, 23 aa, Homo sapiens
QGPMGPMGPRGPPGPAGAPGPQG

SEQ ID NO: 118 - protein, 31 aa, Homo sapiens
QGPRGLPGTPGTDGPKGASGPAGPPGAQGPP

SEQ ID NO: 119 - protein, 28 aa, Homo sapiens
RSGETGPAGPPGNPGPPGPPGPPGPGID

SEQ ID NO: 120 - protein, 21 aa, Homo sapiens
RVGPPGSNGNPGPPGPPGPSG

SEQ ID NO: 121 - protein, 14 aa, Homo sapiens
SNGNPGPPGPPGPS

SEQ ID NO: 122 - protein, 9 aa, Homo sapiens
SPGPMGPRG

SEQ ID NO: 123 - protein, 21 aa, Homo sapiens
VKGESGSPGENGSPGPMGPRG

SEQ ID NO: 124 - protein, 10 aa, Homo sapiens
LTGPAGGGGC

SEQ ID NO: 125 - protein, 10 aa, Homo sapiens
LTGPAGEPGK

SEQ ID NO: 126 - protein, 11 aa, Homo sapiens
GLTGPAGEPGK

SEQ ID NO: 127 - protein, 10 aa, Homo sapiens
KGATGPLGPK

SEQ ID NO: 128 - protein, 10 aa, Homo sapiens
CGGGRDGAAG

(19) United States
(12) Patent Application Publication
(10) Pub. No.: US 2011/0244483 A1
(43) Pub. Date: Oct. 6, 2011

(54) ASSESSMENT OF PROTEIN DEGRADATION BY MEASUREMENT OF COLLAGEN FRAGMENTS

(75) Inventors: Diana J. Leeming, Copenhagen (DK); Inger Byrjalsen, Hoersholm (DK); Per Qvist, Klampenborg (DK); Morten A. Karsdal, Copenhagen (DK)

(73) Assignee: NORDIC BIOSCIENCE A/S

(21) Appl. No.: 13/128,939

(22) PCT Filed: Nov. 11, 2009
(86) PCT No.: PCT/EP2009/064997

§ 371 Date: Jun. 23, 2011

(30) Foreign Application Priority Data
Nov. 13, 2008 (GB) 0820786.2

(51) Int. Cl. G01N 33/566; C07K 16/00

(52) U.S. Cl. 435/7.9; 436/501; 530/387.1

(57) ABSTRACT

A method of assay measuring in a biological sample fragments of a protein that contain an N-terminal neo-epitope and a C-terminal neo-epitope, each generated by protease cleavage of said protein, comprises binding the N-terminal neo-epitope with a first specific antibody and binding the C-terminal neo-epitope with a second specific antibody, and detecting the extent of dual binding of said antibodies.

The present invention relates to the measurements in body fluids of certain peptide fragments generated by proteolytic cleavage of type II collagen using two antibodies each recognising a neo-epitope located on said peptide fragment for quantitative assessment of articular cartilage degradation.

Osteoarthrtis is one of the leading causes of disability in the world, with more than 10% of the elderly population having symptomatic disease (Woolf & Pfleger, 2003). The incidence increases with age, and by age 65, 80% has radiographic evidence of OA (Lawrence et al., 1998). Therefore, osteoarthritis is both prevalent and a serious burden to the patient and the society. However, at present there is little to offer the affected individuals for prevention of the disease or treatment in the early stages. For many patients, hip or knee replacement is eventually the only treatment option.

Although the pathogenicity of osteoarthritis is not fully understood at present, it is evident that a central hallmark in this slow, chronic disease is progressive destruction of the articular joints, which consist of bone, cartilage and the synovium. In particular the cartilage has attracted much attention, and the different grades and stages of OA cartilage histopathology have recently been detailed described by a working group under OARSI (Pritzker et al., 2006). This system encompasses 7 grades (or severity levels) with involvement of the deeper cartilage layers in more advanced OA disease, and when combined with the extent of cartilage involvement expressed in 5 stages this leads to a semi-quantitative scoring system of 0 to 24.

However, the lack of sensitive, specific and fast analytical techniques to assess important metabolic processes in articular cartilage and their effects on the structure of the tissue is a major barriere to effective drug development in OA.

In particular, major efforts have been allocated to the development of new and better biochemical markers of cartilage turnover.

Cartilage, including articular cartilage, is for the most part composed of collagen type II (60%-70% of dry weight) and proteoglycans (10% of dry weight). Cartilage degradation is mainly mediated by the MMPs and the closely related ADAM-TS (a disintegrin and metalloproteinase with thrombospondin motifs), but collagen type II is most sensitive to MMP activity (Dean et al., 1989; Reboul et al., 1996; Hui et al., 2003). The action of these proteases results in the release of various extracellular fragments which could be candidates as biomarkers of cartilage degradation.

Proteolytic cleavage of type II collagen has been reported in numerous studies, and several different degradation fragments of collagen type II have been indicated as useful for monitoring degenerative diseases of the cartilage (Schaller et al., 2005; Sumer et al., 2006; Birmingham et al., 2006).

A fragment of the C-telopeptide of type II collagen, i.e. CTX-II, is generated by MMP-activity (Christgau et al., 2001; Mouritzen et al., 2003) and measurement of CTX-II has been reported for monitoring degradation of type II collagen in experimental setups assessing cartilage degradation (Schaller et al., 2005) as well as in humans (Reijman et al., 2004).

However, the first report of using antibodies for detection of collagen type II fragments came from Billinghurst and co-workers (Billinghurst et al., 1997), who described detection of an amino-terminal neoepitope on the shorter fragment of type II collagen after cleavage by collagenase.

More well-described in the literature is the C2C neoepitope at the C-terminus of the ¾ length fragment (Fraser et al., 2003; Poole et al., 2004). This test is dependant of the binding of a monoclonal antibody to the amino acid sequence EGPP(OH)GPQG SEQ ID NO:1 (Poole et al., 2004).

Other tests for collagen type II fragments include the C1,2C, which, however, is based on the amino acid sequence GPP(OH)GPQG SEQ ID NO:2 found in both type I and type II (Billinghurts et al., 1997). Also, another fragment generated by the action of collagenase is the TIINE fragment (Otterness et al., 1997) and can be detected using monoclonal antibody 9A4 recognising the neoepitope Gly-Pro-Pro-Gly-Pro-Gln-Gly-COOH SEQ ID NO:3. Combined with monoclonal antibody 5109 (Downs et al., 2001) as a capture antibody, the sandwich test is claimed to be specific for type II collagen fragments. Monoclonal antibody 5109 binds to the internal amino acid sequence GEPGDDAPS SEQ ID NO:20 and the sandwich test detects collagen fragments of variable length at the N-terminus of the binding sequence of monoclonal antibody 5109.

Zhen (2008) and co-workers discloses identification and characterization of proteolytic peptide products of human articular cartilage, including type II collagen fragments.

Cathepsin K cleavage of type II collagen has been reported in a few studies. The first study to associate cathepsin K with cleavage of type II collagen was reported by Kafienah et al. (1998). Kafienah and coworkers described helical cleavage site(s) of collagen type II by Cathepsin K, and provided the amino acid sequence for one cleavage site, i.e.

SEQ ID NO: 5
PGDDGEAGKPG [symbol] KSGERGPPG (bovine sequence)

Ten years after, Dejica et al. (2008) reported the development of an enzyme-linked immunosorbent assay based on polyclonal antibodies against the C-terminal neoepitope (C2K) of the cathepsin K cleavage site reported by Kefienah, i.e.

| | |
|---|---|
| PGDDGEAGKPG [symbol] KAGERGPPG | SEQ ID NO: 6 |

As the seven C-terminal amino acids of this epitope, i.e. GEAGKPG SEQ ID NO:20 can be found in type I collagen as well, this test will not distinguish type I and type II collagen fragments generated by cathepsin K activity. In contrast, according to the present invention such sequences are de-selected to increase specificity for type II collagen fragments.

U.S. Pat. No. 6,642,007 (Saltarelli) disclose methods for monitoring urine for type II collagen fragment using a combination of a capture antibody and a detection antibody, such that type II collagen is distinguished from other collagen fragments.

U.S. Pat. No. 6,030,792 (Otterness) discloses antibodies for detecting collagen type II fragments resulting from collagenase cleavage. In particular, the following sequences are disclosed;

| | |
|---|---|
| GPPGPQG | SEQ ID NO: 7 |
| GEPGDDGPSG | SEQ ID NO: 8 |
| APGEDGRPGPPGP | SEQ ID NO: 9 |
| GKVGPSGAPGEDGRPG | SEQ ID NO: 10 |
| AEGPPGPQG | SEQ ID NO: 11 |
| GPPGPQGLAG | SEQ ID NO: 12 |
| GEPGDDGPS | SEQ ID NO: 13 |
| GEPGDDGPSGAEGPPG | SEQ ID NO: 14 |
| EKGEPGDDAPSGAEGPPGPQG | SEQ ID NO: 15 |
| GPPGPPGKPGDDGEAGKPGKA | SEQ ID NO: 16 |
| GPPGPRGRSGETGPAGPPGNP | SEQ ID NO: 17 |
| GAPGPQGFQGNPGEPGEPGVSY | SEQ ID NO: 18 |
| GEPGDDAGPSGAEGPPGPQG | SEQ ID NO: 19 |

A series of patents (U.S. Pat. Nos. 6,602,980; 6,566,492; 6,348,320; 6,255,056; 6,153,732; 6,143,511; 6,100,379; 5,919,634; 5,702,909; 5,688,652; 5,641,837; 5,641,687; 5,532,169) (Eyre) relates to peptides and methods for cartilage resorption assays employing antibodies binding to epitopes in the telopeptides of type II collagen.

U.S. Pat. No. 5,283,197 (Robins) describes methods of detecting collagen fragments cross-linked with lysyl pyridinoline or hydroxylysyl pyridinoline.

U.S. Pat. No. 7,410,770 (Reginster) disclosed methods for detection of collagenase-generated fragments of collagen type II using antibody binding to epitope in the amino acid sequence HRGYPGLDG SEQ ID NO: 23 located in the helical region of collagen type II.

U.S. Pat. No. 6,132,976 (Poole) discloses methods for detecting cartilage degradation using antibodies which does not bind to unwound (native) type II collagen fragments but only to fragments being generated by collagenase cleavage. In particular the following amino acid sequences originating from type II collagen are included;

| | |
|---|---|
| CGKVGPSGAPGEDGRPGPPGPQY | SEQ ID NO: 21 |
| APGEDGRPGPPGP | SEQ ID NO: 22 |
| GQPG | SEQ ID NO: 24 |
| GPPGPQG | SEQ ID NO: 7 |
| CGGEGPPGPQG | SEQ ID NO: 25 |
| GAEGPPGPQGLAGQRGIVG | SEQ ID NO: 26 |
| GAPGTPGPQGIAGQRGVVG | SEQ ID NO: 27 |
| GPPGTPGPQGLLGAPGILG | SEQ ID NO: 28 |
| GPPGAPGPLGIAGITGARG | SEQ ID NO: 29 |
| CGGEGPPGPQGL | SEQ ID NO: 30 |
| CGGEGPPGPQGLA | SEQ ID NO: 31 |
| CGGEGPPGPQ | SEQ ID NO: 32 |
| CGGEGPPGP | SEQ ID NO: 33 |
| CGPPGPQG | SEQ ID NO: 34 |

U.S. Pat. No. 7,115,378 (Welsch) describes the use of mass spectrometry for identifying and quantifying peptides resulting from enzyme cleavage of collagen type II. The technique is used for identification and quantification of the peptides in a biological sample to assess activity of proteolytic enzymes in osteoarthritis and rheumatoid arthritis. In particular, the following sequences originating from humans are disclosed;

SEQ ID NO: 7
GPPGPQG
SEQ ID NO: 35
LQGPAGPPGEKGEPGDDGPSGAEGPPGPQG
PQG
SEQ ID NO: 36
PGPQG
SEQ ID NO: 37
PPGPQG
SEQ ID NO: 38
VLQGPAGPPGEKGEPGDDGPSGAEGPPGPQG
SEQ ID NO: 39
KGARGDSGPPGRAGEPGLQGPAGPPGEKGEPGDDGPSGAEGPPGPQG
SEQ ID NO: 40
ARGDSGPPGRAGEPGLQGPAGPPGEKGEPGDDGPSGAEGPPGPQG
SEQ ID NO: 41
GPAGPPGEKGEPGDDGPSGAEGPPGPQG
SEQ ID NO: 42
GPIGPPGERGAPGNRGFPGQDGLAGPKGAPGERGPSGLAGPKGANGDPGR
PGEPGLPGARGLTGRPGDAGPQGKVGPSGAPGEDGRPGPPGPQGARGQPG
VMGFPGPKGANGEPGKAGEKGLPGAPGLGLPGKDGETGAEGPPPA......

U.S. Pat. No. 6,706,490 (Cook) describes the detection of antibodies to collagen using CB peptides, in particular CB10, of mammalian type II collagen. Cyanogen bromide cleaves the carboxyl terminal of methionine residues thereby producing the CB peptides. The CB10 peptide has the sequence:

SEQ ID NO: 43
MPGERGAAGIAGPKGDRGDVGEKGPEGAPGKDGGRGLTGPIGPPGPAGAN
GEKGEVGPPGPAGSAGARGAPGERGETGPPGTSGIAGPPGADGQPGAKGE
QGEAGQKGDAGAPGPQGPSGAPGPQGPTGVTGPKGARGAQGPPGATGFPG
AAGRVGPPGSNGNPGPPGPPGPSGKDGPKGARGDSGPPGRAGEPGLQGPA
GPPGEKGEPGDDGPSGAEGPPGPQGLAGQRGIVGLPGQRGERGFPGLPGP
SGEPGQQGAPGASGDRGPPGPVGPPGLTGPAGEPGREGSPGADGPPGRDG
AAGVKGDRGETGAVGAPGAPGPPGSPGPAGPTGKQGDRGEAGAQGPM

U.S. Pat. No. 7,195,883 (Rosenquist) disclose sandwich immunoassays in which a single antibody specific for the amino acid sequence EKGPDP SEQ ID NO:44 is used to detect telopeptide fragments of type II collagen.

U.S. Pat. Nos. 6,420,125 and 6,107,047 (Fledelius) disclose methods of measuring the rate of degradation of collagen using antibodies binding to an amino acid sequence of type II collagen containing an isoaspartic acid residue. Also, the use of synthetic peptides having an amino acid sequence of type II collagen that contains an isoaspartic acid residue is described. In particular, the following sequences from type II collagen are described;

| | |
|---|---|
| GDIK*DIV | SEQ ID NO: 45 |
| EKGP*D, | SEQ ID NO: 46 |

where (*) denotes an isomerised peptide bond.

U.S. Pat. No. 6,300,083 (Fledelius) describes the determination of the amount of a D-amino acid containing fragment of the protein in a body fluid using an antibody capable of discriminating between the D-amino acid containing fragment and its L-amino acid containing analogue. In particular, the application includes the following peptide sequences from type II collagen;

| | |
|---|---|
| GDIKDIV | SEQ ID NO: 47 |
| EKGPD | SEQ ID NO: 48 |

U.S. Pat. Nos. 6,372,442 and 6,210,902 (Bonde) describes methods of characterizing the degradation of type II collagen. At least two distinct immunological assays should be used, each using a different immunological binding partner, and a numerical index is formed representing the difference in the results of the assays. In particular, the following type II collagen sequences are disclosed;

| | |
|---|---|
| EKGPDP | SEQ ID NO: 44 |
| EKGPD | SEQ ID NO: 48 |
| GVK | |
| PGVKG | SEQ ID NO: 49 |
| PGPKGE | SEQ ID NO: 50 |
| GQKGEP | SEQ ID NO: 51 |
| GDIKDIV | SEQ ID NO: 47 |

U.S. Pat. Nos. 6,355,442; 6,342,361; 6,323,314 and 6,110,689 (Qvist) describe the use of antibodies recognizing synthetic peptides for detection of collagen fragments. In particular, the following amino acid sequences are included;

| | |
|---|---|
| PGPKGE | SEQ ID NO: 50 |
| GQKGEP | SEQ ID NO: 51 |
| GDIKDIV | SEQ ID NO: 47 |
| EKGPD | SEQ ID NO: 48 |
| GVK | |
| PGVKG | SEQ ID NO: 49 |

U.S. Pat. No. 6,010,863 (Te Koppele) discloses the use of a sandwich immunoassay for the detection of collagen degradation using a first antibody directed at an epitope present on a collagen molecule at a distance of up to 165 amino acids from a collagen telopeptide crosslink site, and a second antibody directed at another epitope of the crosslinked collagen molecule.

U.S. Pat. No. 5,541,295 (Barrack) discloses monoclonal antibodies which bind specifically to Type II collagen, but not to its peptides, or vice versa. In particular, the following type II collagen sequences are included;

| | |
|---|---|
| GFQGL-Xaa-G-Xaa-Xaa-G-Xaa-Xaa-G | SEQ ID NO: 52 |
| GLQGL-Xaa-G-Xaa-Xaa-G-Xaa-SG | SEQ ID NO: 53 |

None of the above mentioned patents disclose the use of two antibodies each of which binds to a neo-epitope on the same collagen type II fragment.

U.S. Pat. No. 6,642,007 (Saltarelli) uses two antibodies in a sandwich, however, none of them are required to bind specifically to neo-epitopes. The capture antibody is requested to bind to type II collagen fragments, to the substantial exclusion of any binding to type I or III collagen fragments, and the other antibody, i.e. the detector antibody, should bind specifically to collagenase-generated collagen fragments. Specificity to the amino acid sequence in the cleavage site, i.e. the neo-epitope, is, however, not required.

Also, U.S. Pat. No. 7,195,883 (Rosenquist) discloses sandwich immunoassays for type II collagen, however using a single antibody. According to the present invention, two antibodies with different epitope-specificity are required, i.e. one for each neo-epitope.

The present invention now provides a method of assay, comprising measuring in a biological sample fragments of a protein that contain an N-terminal neo-epitope and a C-terminal neo-epitope, each said neo-epitope being an amino acid sequence generated by protease cleavage of said protein, said method comprising binding the N-terminal neo-epitope with a first immunological binding partner specific for the presence of said N-terminal neo-epitope and binding the C-terminal neo-epitope with a second immunological binding partner specific for the presence of said C-terminal neo-epitope, and detecting the extent of dual binding of said binding partners.

The biological sample may in particular be a body fluid sample and may be blood, serum, plasma, or urine.

Said assay may be performed as a sandwich assay in which one of said immunological binding partners is immobilised to a solid support, said fragments are bound to said immobilised antibody and the binding of the other of said immunological binding partners to said fragments is detected. The sandwich assay may be performed as a homogeneous sandwich assay or as a heterogeneous sandwich assay.

Preferably, neither said first nor said second immunological binding partner specifically binds the intact protein from which said fragments derive and preferably neither said first nor said second immunological binding partner specifically binds fragments of said protein containing the amino acid sequence of its respective said neo-epitope extended beyond the protease cleavage site.

Preferably, said neo-epitopes are from collagen type II, whereby the assay may be diagnostic of cartilage degeneration and may provide a diagnostic index in respect of arthritis.

Said neo-epitopes may be produced by cleavage of collagen type II by an MMP or by cathepsin K.

Preferably, the first immunological binding partner is specific for an epitope defined by one of the following collagen type II cleavage amino acid sequences:

| | |
|---|---|
| [symbol] DQGVPG . . .; | SEQ ID NO: 54 |
| [symbol] REGSPG . . .; | SEQ ID NO: 55 |
| *LAGPKG . . .; | SEQ ID NO: 56 |
| and | |
| * [symbol] LTGPAG . . .. | SEQ ID NO: 57 |

Preferably, the second immunological binding partner is specific for an epitope defined by one of the following collagen type II cleavage amino acid sequences:

| | |
|---|---|
| . . . PKGARG [symbol] ; | SEQ ID NO: 58 |
| . . . GQPGPA [symbol] ; | SEQ ID NO: 59 |
| . . . EPGGVG [symbol] ; | SEQ ID NO: 60 |
| and | |
| . . . RDGAAG [symbol] . | SEQ ID NO: 61 |

Preferably, said first and second immunological binding partners are in combination specifically reactive with a collagen type II peptide fragment of the sequence:

| | |
|---|---|
| LTGPAGEPGREGSPGADGPPGRDGAAG | SEQ ID NO: 62 |
| or | |
| REGSPGADGPPGRDGAAG | SEQ ID NO: 63 |

Optionally, severity of arthritis development in the subject is evaluated by comparing the level of binding measured in said assay with levels previously established in healthy subjects and or in subjects having pathological arthritis activity.

The invention includes an immunological assay kit comprising a first immunological binding partner specific for an epitope containing an isomerised amino acid residue and a second immunological binding partner specific for a protease generated neo-epitope. Said kit may further comprise at least one of calibration standards immunoreactive with said binding partners, a wash reagent, a buffer, a secondary immunological binding partner for revealing binding between said first or second immunological binding partner and components of a sample, an enzyme label, an enzyme label substrate, a stopping reagent, and instructions for conducting an assay using said kit.

Assay formats useful in accordance with the present invention include both heterogeneous and homogeneous sandwich assay formats.

Homogeneous formats include the use of two different immunological binding partners bound to respective beads wherein the beads incorporate a detectable proximity signal activated when the beads are brought into proximity by their respective binding partners both binding to sites on a single fragment molecule.

Heterogeneous assay formats include those in which one of said immunological binding partners is immobilised to a solid support, said fragments are bound to said immobilised antibody and the binding of the other of said immunological binding partners to said fragments is detected.

Immunological binding partners for use in the present invention include whole antibodies, especially monoclonal antibodies, and antibody fragments with specific binding affinity. These include binding fragments such as Fab or F(ab′)₂.

Assays according to the invention are useful in diagnosis or disease progression monitoring in respect of pathological conditions which lead to the release of fragments detectable in said assays. The disease indicated will depend on the fragments measured. Of especial interest for diagnosis or monitoring of osteoarthritis is collagen II. Other diseases involving destruction of cartilage could also be diagnosed and monitored with the tests according to the present invention. Such disease include rheumatoid arthritis.

Examples of neoepitopes of proteolytic cleavage of collagen type II include Cathepsin K generated neoepitopes and MMP-generated neoepitopes. Also, included are fragments generated by proteolytic cleavage of type II collagen by aggrecanases, e.g. ADAM-TS4 and ADAM-TS5. The identification of collagen type II sequences carrying Cathepsin K and MMP9 neopitopes could be performed as described below. In a similar manner collagen type II sequences carrying other MMP or aggrecanase generated neoepitopes could be identified, or MMP generated neoepitopes could be based on publicly available information on MMP generated peptide products of human articular cartilage (then et al. Arthritis Rheum 2008 58(8):2420-31). To ascertain protein and protease specificity, preferred biomarker neoepitopes of cleavage sites may be selected as follows.

Human collagen type II (BIOCOL BC-3001) was dissolved in 10 mM acetic acid (400 μl added to 1 mg of collagen type II). Ten μg of procathepsin K (Calbiochem 342001) was activated by addition of 200 μl of 100 mM sodium acetate containing 10 mM DTT and 5 mM EDTA, pH 3.9 for 40 minutes at room temperature. Ten μg of MMP9 (Calbiochem 444231) was activated by addition of 200 μl of 1 mM APMA in DMSO for 2 hours at 37° C. For the Cathepsin K cleavage, 60 μl of collagen type II was added 120 μl of 50 mM sodium acetate, pH 5.5 containing 20 mM L-cystein and 24 μl of activated cathepsin K for 4 hours at 37° C. For the MMP9 cleavage, 60 μl of collagen type II was added 120 μl of 100 mM Tris-HCl, 100 mM sodium chloride, 10 mM calcium chloride, 2 mM zinc chloride, pH 8.0 and 20 μl of MMP9 for 3 days at 37° C. The resulting proteolytic cleavage fragments were characterized by high performance liquid chromatography (HPLC)-tandem mass spectrometry (MS/MS) analysis. The MS/MS spectra were searched against protein databases using Sequest and X! Tandem database search algorithms. The following sequence hits of fragments were found for the Cathepsin K cleaved collagen type II:

| | |
|---|---|
| AQGPPGATGFPGAAGR | SEQ ID NO: 64 |
| ASGDRGPPGPV | SEQ ID NO: 65 |
| ASGDRGPPGPVGPPG | SEQ ID NO: 66 |
| GANGEKGEVGPPGPA | SEQ ID NO: 67 |
| GAPGEDGRPGPPGPQ | SEQ ID NO: 68 |
| GARGAPGERGETGPPGPA | SEQ ID NO: 69 |
| GDRGPPGPV | SEQ ID NO: 70 |
| GERGFPG | SEQ ID NO: 71 |
| GERGFPGER | SEQ ID NO: 72 |
| GESGSPGENGSPGPM | SEQ ID NO: 73 |
| GLPGPPGPPGEGGKPG | SEQ ID NO: 74 |
| GPIGPPGPA | SEQ ID NO: 75 |
| GPPGPPGKPGDDGEAGKPG | SEQ ID NO: 76 |
| GPPGPV | SEQ ID NO: 77 |
| GPPGPVGPA | SEQ ID NO: 78 |
| LPGPPGPPGEGGKPG | SEQ ID NO: 79 |
| NPGPPGPPGPPGPG | SEQ ID NO: 80 |
| PIGPP | SEQ ID NO: 81 |
| REGSPGADGPPGRDGAAGVK | SEQ ID NO: 82 |
| SNGNPGPPGPPGPS | SEQ ID NO: 83 |

Identified Cathepsin K-generated fragments were aligned with the sequence for human collagen type II (sp|P02458|CO2A1_HUAN Collagen alpha-1(II) chain), and cleavage sites were localized as indicated by the arrows.

SEQ ID NO: 84
QMAGGFDEKAGGAQLGVMQGPMGPMGPRGPPGPAGAPGPQGFQGNPG
EPGEPGVSGPMGPR [symbol] GPPGPPGKPGDDGEAGKPG [symbol] KAGERGPPGPQG
ARGFPGTPGLPGVKGHRGYPGLDGAKGEAGAPGVK [symbol] GESGSPGENGSP
GPM [symbol] GPRGLPGERGRTGPAGAAGARGNDGQPGPA [symbol] GPPGPV [symbol] GPA [symbol]
GGPGFPGAPGAKGEAGPTGARGPEGAQGPRGEPGTPGSPGPAGASGN
PGTDGIPGAKGSAGAPGIAGAPGFPGPRGPPGPQGATGPLGPKGQTGEPG
IAGFKGEQGPKGEPGPAGPQGAPGPAGEEGKRGARGEPGGVG [symbol] PIGPP [symbol]
GERGAPGNRGFPGQDGLAGPKGAPGERGPSGLAGPKGANGDPGRPGE
PGLPGARGLTGRPGDAGPQGKVGPS [symbol] GAPGEDGRPGPPGPQ [symbol] GARGQ
PGVMGFPGPKGANGEPGKAGEKGLPGAPGLRGLPGKDGETGAAGPPGPAG
PAGERGEQGAPGPSGFQ [symbol] G [symbol] LPGPPGPPGEGGKPG [symbol] DQGVPGEAG
APGLVGPR [symbol] GERGFPG [symbol] ER [symbol] GSPGAQGLQGPRGLPGTPGTDGPKG
ASGPAGPPGAQGPPGLQGMPGERGAAGIAGPKGDRGDVGEKGPEGAPGKD
GGRGLT [symbol] GPIGPPGPA [symbol] GANGEKGEVGPPGPA [symbol] GSA [symbol] GARGAPG
ERGETGPPGPA [symbol] GFAGPPGADGQPGAKGEQGEAGQKGDAGAPGPQGPS
GAPGPQGPTGVTGPKGARG [symbol] AQGPPGATGFPGAAGR [symbol] VGPPG [symbol] SN
GNPGPPGPPGPS [symbol] GKDGPKGARGDSGPPGRAGEPGLQGPAGPPGEKGE
PGDDGPSGAEGPPGPQGLAGQRGIVGLPGQRGERGFPGLPGPSGEPGKQG
APG [symbol] AS [symbol] GDRGPPGPV [symbol] GPPG [symbol] LTGPAGEPG [symbol] REGSPGADGP
PGRDGAAGVK [symbol] GDRGETGAVGAPGAPGPPGSPGPAGPTGKQGDRGEAG
AQGPMGPSGPAGARGIQGPQGPRGDKGEAGEPGERGLKGHRGFTGLQGLP
GPPGPSGDQGASGPAGPSGPRGPPGPVGPSGKDGANGIPGPIGPPGPRGR
SGETGPAGPPG [symbol] NPGPPGPPGPPGPG [symbol] IDMSAFAGLGPREKGPDPLQ
YMRA

The following sequence hits of fragments were found for the MMP9 cleaved collagen type II:

| | |
|---|---|
| AAGARGNDGQPGPAGPPGPVGPA | SEQ ID NO: 85 |
| AQGPRGEPGTPGSPGPAG | SEQ ID NO: 86 |
| ARGAPGERGETGPPGPAG | SEQ ID NO: 87 |
| ASGDRGPPGPV | SEQ ID NO: 88 |
| ASGDRGPPGPVG | SEQ ID NO: 89 |
| ATGPLGPKG | SEQ ID NO: 90 |
| DRGPPGPVGPPG | SEQ ID NO: 91 |
| ERGAPGNRGFPGQDGLAGPKGAPGERGPSG | SEQ ID NO: 92 |
| FQGLPGPPGPPGEGGKPGDQGVPGEAGAPGLVGPR | SEQ ID NO: 93 |
| FQGLPGPPGPPGEGGKPGDQGVPGEAGAPGLVGPRG | SEQ ID NO: 94 |
| FTGLQGLPGPPGPSG | SEQ ID NO: 95 |
| FTGLQGLPGPPGPSGDQGASGPAGPSGPRGPPGPVG | SEQ ID NO: 96 |
| PSG | |
| GANGEKGEVGPPGPA | SEQ ID NO: 97 |
| GAPGEDGRPGPPGPQ | SEQ ID NO: 98 |
| GAPGEDGRPGPPGPQG | SEQ ID NO: 99 |
| GAPGERGETGPPGPA | SEQ ID NO: 100 |
| GESGSPGENGSPGPM | SEQ ID NO: 101 |
| GPIGPPGPA | SEQ ID NO: 102 |
| GPPGPV | SEQ ID NO: 103 |
| GPPGPVGPPG | SEQ ID NO: 104 |
| GPRGPPGPAGAPGPQG | SEQ ID NO: 105 |
| GVMQGPMGPMGPRGPPGPAGAPGPQG | SEQ ID NO: 106 |
| IVGLPGQRGERGFPGLPGPSGEPGK | SEQ ID NO: 107 |
| KQGDRGEAGAQGPMGPSGPAG | SEQ ID NO: 108 |
| KVGPSGAPGEDGRPGPPGPQG | SEQ ID NO: 109 |
| LPGKDGETGAAGPPGPAGPAG | SEQ ID NO: 110 |
| LPGKDGETGAAGPPGPAGPAGERGEQGAPGPSG | SEQ ID NO: 111 |
| LQGLPGPPGPSGDQGASGPAGPSGPRGPPGPVGPSG | SEQ ID NO: 112 |
| LTGPAGEPGREGSPGAD | SEQ ID NO: 113 |
| LTGPAGEPGREGSPGADGPPGRDGAAG | SEQ ID NO: 62 |
| LTGPIGPPGPAG | SEQ ID NO: 115 |
| LTGRPGDAGPQGKVGPSGAPGEDGRPGPPGPQG | SEQ ID NO: 116 |
| QGPMGPMGPRGPPGPAGAPGPQG | SEQ ID NO: 117 |
| QGPRGLPGTPGTDGPKGASGPAGPPGAQGPP | SEQ ID NO: 118 |
| RSGETGPAGPPGNPGPPGPPGPPGPGID | SEQ ID NO: 119 |
| RVGPPGSNGNPGPPGPPGPSG | SEQ ID NO: 120 |
| SNGNPGPPGPPGPS | SEQ ID NO: 121 |
| SPGPMGPRG | SEQ ID NO: 122 |
| VKGESGSPGENGSPGPMGPRG | SEQ ID NO: 123 |

Identified MMP9-generated fragments were aligned with the sequence for human collagen type II (sp|P02458|CO2A1_HUAN Collagen alpha-1(II) chain), and cleavage sites were localized as indicated by the stars.

SEQ ID NO: 84
QMAGGFDEKAGGAQL*GVM*QGPMGPM*GPRGPPGPAGAPGPQG*FQ
GNPGEPGEPGVSGPMGPRGPPGPPGKPGDDGEAGKPGKAGERGPPGP
QGARGFPGTPGLPGVKGHRGYPGLDGAKGEAGAPG*VK*GESGSPGE
NG*SPGPM*GPRG*LPGERGRTGPAG*AAGARGNDGQPGPAGPPGPV
GPA*GGPGFPGAPGAKGEAGPTGARGPEG*AQGPRGEPGTPGSPGPA
G*ASGNPGTDGIPGAKGSAGAPGIAGAPGFPGPRGPPGPQG*ATGPL
GPKG*QTGEPGIAGFKGEQGPKGEPGPAGPQGAPGPAGEEGKRGARG
EPGGVGPIGPPG*ERGAPGNRGFPGQDGLAGPKGAPGERGPSG*LAG
PKGANGDPGRPGEPGLPGARG*LTGRPGDAGPQG*KVGPS*GAPGED
GRPGPPGPQ*G*ARGQPGVMGFPGPKGANGEPGKAGEKGLPGAPGLR
G*LPGKDGETGAAGPPGPAGPAG*ERGEQGAPGPSG*FQGLPGPPGP
PGEGGKPGDQGVPGEAGAPGLVGPR*G*ERGFPGERGSPGAQGL*QG
PRGLPGTPGTDGPKGASGPAGPPGAQGPP*GLQGMPGERGAAGIAGP
KGDRGDVGEKGPEGAPGKDGGRG*LT*GPIGPPGPA*G*ANGEKGEV
GPPGPA*GSAG*AR*GAPGERGETGPPGPA*G*FAGPPGADGQPGAK
GEQGEAGQKGDAGAPGPQGPSGAPGPQGPTGVTGPKGARGAQGPPGA
TGFPGAAG*RVGPPG*SNGNPGPPGPPGPS*G*KDGPKGARGDSGPP
GRAGEPGLQGPAGPPGEKGEPGDDGPSGAEGPPGPQGLAGQRG*IVG
LPGQRGERGFPGLPGPSGEPGK*QGAPG*ASG*DR*GPPGPV*G*PP
G*LTGPAGEPGREGSPGAD*GPPGRDGAAG*VKGDRGETGAVGAPGA
PGPPGSPGPAGPTG*KQGDRGEAGAQGPMGPSGPAG*ARGIQGPQGP
RGDKGEAGEPGERGLKGHRG*FTG*LQGLPGPPGPSG*DQGASGPAG
PSGPRGPPGPVGPSG*KDGANGIPGPIGPPGPRG*RSGETGPAGPPG
NPGPPGPPGPPGPGID*MSAFAGLGPREKGPDPLQYMRA

Cleavage sites were localized as indicated by arrows for the Cathepsin K-generated and stars for MMP9-generated sites.

SEQ ID NO: 84
QMAGGFDEKAGGAQL*GVM*QGPMGPM*GPRGPPGPAGAPGPQG*FQ
GNPGEPGEPGVSGPMGPR [symbol] GPPGPPGKPGDDGEAGKPG [symbol] KAGERGPPG
PQGARGFPGTPGLPGVKGHRGYPGLDGAKGEAGAPG*VK* [symbol] GESGSPG
ENG*SPGPM* [symbol] GPRG*LPGERGRTGPAG*AAGARGNDGQPGPA [symbol] GPP
GPV [symbol] GPA* [symbol] GGPGFPGAPGAKGEAGPTGARGPEG*AQGPRGEPGTPG
SPGPAG*ASGNPGTDGIPGAKGSAGAPGIAGAPGFPGPRGPPGPQG*
ATGPLGPKG*QTGEPGIAGFKGEQGPKGEPGPAGPQGAPGPAGEEGK
RGARGEPGGVG [symbol] PIGPP [symbol] G*ERGAPGNRGFPGQDGLAGPKGAPGERG
PSG*LAGPKGANGDPGRPGEPGLPGARG*LTGRPGDAGPQG*KVGPS* [symbol]
GAPGEDGRPGPPGPQ* [symbol] G*ARGQPGVMGFPGPKGANGEPGKAGEKGL
PGAPGLRG*LPGKDGETGAAGPPGPAGPAG*ERGEQGAPGPSG*FQ [symbol] G [symbol]
LPGPPGPPGEGGKPG [symbol] DQGVPGEAGAPGLVGPR* [symbol] G*ERGFPG [symbol] E
R [symbol] GSPGAQGL*QGPRGLPGTPGTDGPKGASGPAGPPGAQGPP*GLQGMP
GERGAAGIAGPKGDRGDVGEKGPEGAPGKDGGRG*LT* [symbol] GPIGPPGPA* [symbol]
G*ANGEKGEVGPPGPA* [symbol] GSA [symbol] G*AR*GAPGERGETGPPGPA* [symbol]
G*FAGPPGADGQPGAKGEQGEAGQKGDAGAPGPQGPSGAPGPQGPTGV
TGPKGARG [symbol] AQGPPGATGFPGAAG*R [symbol] VGPPG* [symbol] SNGNPGPPGPPG
PS* [symbol] G*KDGPKGARGDSGPPGRAGEPGLQGPAGPPGEKGEPGDDGPSGA
EGPPGPQGLAGQRG*IVGLPGQRGERGFPGLPGPSGEPGK*QGAPG* [symbol] A
S [symbol] G*DR*GPPGPV* [symbol] G*PPG* [symbol] LTGPAGEPG [symbol] REGSPGAD*GPPG
RDGAAG*VK [symbol] GDRGETGAVGAPGAPGPPGSPGPAGPTG*KQGDRGEAG
AQGPMGPSGPAG*ARGIQGPQGPRGDKGEAGEPGERGLKGHRG*FTG*
LQGLPGPPGPSG*DQGASGPAGPSGPRGPPGPVGPSG*KDGANGIPGP
IGPPGPRG*RSGETGPAGPPG [symbol] NPGPPGPPGPPGPG [symbol] ID*MSAFAGLG
PREKGPDPLQYMRA.

Additional neoepitopes of proteolytic cleavage of collagen type II could be based on publicly available information on protease generated peptide products of human articular cartilage (then et al. Arthritis Rheum 2008 58(8):2420-31).

Any of the possible generated fragments could constitute a biomarker. The biomarker could have cleavage sites at the N-terminal and the C-terminal end of the fragment and no other cleavage sites in between or the biomarker could have cleavage sites at the N-terminal and the C-terminal end and one or more possible cleavage sites in between.

Preferred type II collagen sequences carrying two neoepitopes have been selected as follows.

Preferred biomarker neoepitopes were selected based on protein specificity. The protein specificity of the cleavage sites were assessed by identity search of 6 amino-terminal or 6 carboxy-terminal residues on either site of the cleavage site. The public available programme “Pattinprot” was used in the search of the UNIPROT/SWISSPROT databank. The preferred biomarker neoepitopes of Cathepsin K cleavage sites (arrows) and/or MMP cleavage sites (stars) are indicated by underlined sequences:

SEQ ID NO: 84
QMAGGFDEKAGGAQLGVMQGPMGPMGPRGPPGPAGAPGPQGFQGNPG
EPGEPGVSGPMGPRGPPGPPGKPGDDGEAGKPGKAGERGPPGPQGAR
GFPGTPGLPGVKGHRGYPGLDGAKGEAGAPGVKGESGSPGENGSPGP
MGPRGLPGERGRTGPAGAAGARGNDGQPGPA [symbol] GPPGPVGPAGGPGF
PGAPGAKGEAGPTGARGPEGAQGPRGEPGTPGSPGPAGASGNPGTDG
IPGAKGSAGAPGIAGAPGFPGPRGPPGPQGATGPLGPKGQTGEPGIA
GFKGEQGPKGEPGPAGPQGAPGPAGEEGKRGARGEPGGVG [symbol] PIGPP
GERGAPGNRGFPGQDGLAGPKGAPGERGPSG*LAGPKGANGDPGRPG
EPGLPGARGLTGRPGDAGPQGKVGPSGAPGEDGRPGPPGPQGARGQP
GVMGFPGPKGANGEPGKAGEKGLPGAPGLRGLPGKDGETGAAGPPGP
AGPAGERGEQGAPGPSGFQGLPGPPGPPGEGGKPG [symbol] DQGVPGEAGA
PGLVGPRGERGFPGERGSPGAQGLQGPRGLPGTPGTDGPKGASGPAG
PPGAQGPPGLQGMPGERGAAGIAGPKGDRGDVGEKGPEGAPGKDGGR
GLTGPIGPPGPAGANGEKGEVGPPGPAGSAGARGAPGERGETGPPGP
AGFAGPPGADGQPGAKGEQGEAGQKGDAGAPGPQGPSGAPGPQGPTG
VTGPKGARG [symbol] AQGPPGATGFPGAAGRVGPPGSNGNPGPPGPPGPSG
KDGPKGARGDSGPPGRAGEPGLQGPAGPPGEKGEPGDDGPSGAEGPP
GPQGLAGQRGIVGLPGQRGERGFPGLPGPSGEPGKQGAPGASGDRGP
PGPVGPPG* [symbol] LTGPAGEPG [symbol] REGSPGADGPPGRDGAAG*VKGDRGE
TGAVGAPGAPGPPGSPGPAGPTGKQGDRGEAGAQGPMGPSGPAGARG
IQGPQGPRGDKGEAGEPGERGLKGHRGFTGLQGLPGPPGPSGDQGAS
GPAGPSGPRGPPGPVGPSGKDGANGIPGPIGPPGPRGRSGETGPAGP
PGNPGPPGPPGPPGPGIDMSAFAGLGPREKGPDPLQYMRA.

An additional requirement for preferred biomarker fragments is localization of cleavage sites in proximity of each other, i.e. target fragment should preferably at maximum have a length of 50 amino acid residues, more preferably no more than 40, more preferably no more than 30. Examples of preferred biomarker fragments are:

| | | |
|---|---|---|
| 1 | LTGPAGEPGREGSPGADGPPGRDGAAG | SEQ ID NO: 62 |
| 2 | REGSPGADGPPGRDGAAG | SEQ ID NO: 63 |

For fragment 1, the immunological binding partners should preferably have specific binding affinities for the N-terminal neoepitope of LTGPAG . . . SEQ ID NO:57 and the C-terminal neoepitope of . . . RDGAAG SEQ ID NO:61.

For fragment 2, the immunological binding partners should preferably have specific binding affinities for the N-terminal neoepitope of REGSPG . . . SEQ ID NO:55 and the C-terminal neoepitope of . . . RDGAAG SEQ ID NO:61.

Relevant fragments of other proteins may be defined in a similar way. The invention may be applied to the detection in biological samples of degradation products of other proteins.

The invention will be further described and illustrated by the following examples making reference to the accompanying drawings, in which:

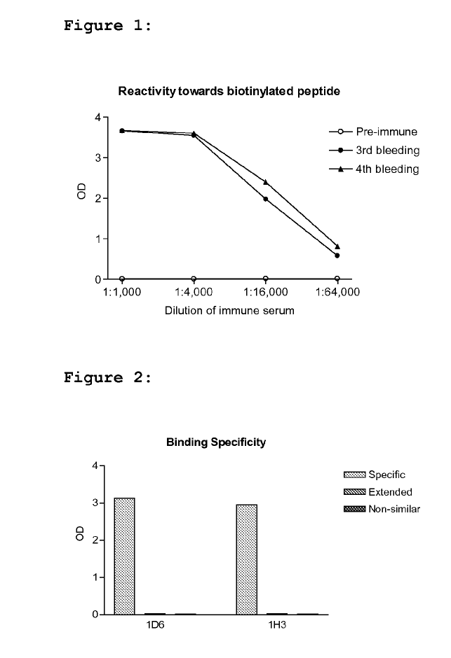

FIG. 1 shows antibody binding in sera studied in Example 1; and

FIG. 2 shows monoclonal antibody binding observed in Example 1.

### EXAMPLE 1

### Generation of Monoclonal Antibodies Recognising Neoepitopes

Synthetic peptides were prepared by standard techniques. To increase immunogenicity, the peptide (LTGPAGGGGC SEQ ID NO:124) was coupled at the C-terminus to the carrier protein KLH using site-directed coupling technology via the cysteine. Before immunisation, the immunogen was mixed 1:1 with Freund's Incomplete Adjuvant and the mixture was injected s.c. in Balb/c mice. The immunisation was repeated every 2 weeks for two months (four immunisations) and then continued with 4 weeks between each immunisation. Blood was obtained from the mice before immunisation initiated and one week after each immunization. The immune response was evaluated by testing the binding reactivity of mouse immune sera towards the C-terminal biotinylated synthetic peptide (LTGPAGEPGK-Biotin SEQ ID NO:125). The test for binding reactivity of mouse immune sera was based on binding of the immune serum to the biotinylated synthetic peptide that was bound to the surface of a streptavidin-coated microtitre plate. After incubation and washing the bound antibody was demonstrated by incubation with anti-mouse IgG conjugated to horseradish peroxidase, washing and addition of the chromogen TMB (FIG. 1).

Subsequent to attaining sufficient immune sera titers in the above mentioned screening test, the selected mice were rested for at least 4 weeks, and boosted i.p. with immunogen without adjuvants. Three days later, the spleen was removed and used for fusion with myeloma cells using standard techniques. Antibodies from growing hybridomas were evaluated by their binding reactivity to the biotinylated synthetic peptide in the assay as described above. Additionally the cleavage specificity of the antibodies was demonstrated by minimum binding reactivity towards a one-residue extended coater (GLTGPAGEPGK-Biotin SEQ ID NO:126) as well as no binding towards a non-similar coater (Biotin-KGATGPLGPK SEQ ID NO:127) (FIG. 2).

### EXAMPLE 2

### Development of Immunoassay for Detection of Collagen Fragments Carrying Two Neo-Epitopes

One monoclonal antibody binding to the N-terminus of the amino acid sequence LTGPAGGGGC SEQ ID NO:124 as described above was biotinylated according to standard procedures and used as capture antibody in a sandwich ELISA. The detector antibody, binding to the C-terminus of the amino acid sequence CGGGRDGAAG SEQ ID NO:128 was labelled with horseradish peroxidase according to standard techniques. Using these two reagents and a microtitre plate coated with streptavidin, the antibodies was pre-diluted in a 10 mM phosphate buffered solution (PBS) with bovine serum albumin (1%) and Tween 20 (0.1%) in a total volume of 100 μL and subsequently incubated in the microtitre plate with 50 μL of human serum sample. A synthetic peptide with the amino acid sequence LTGPAGEPGREGSPGADGPPGRDGAAG SEQ ID NO:62 containing both the N-terminal and C-terminal neo-epitope was used as calibrator. After two hours of incubation the wells were washed and incubated with a chromogen (TMB) for 15 minutes. Subsequently, the colour reaction was stopped by addition of 0.18M sulfuric acid.

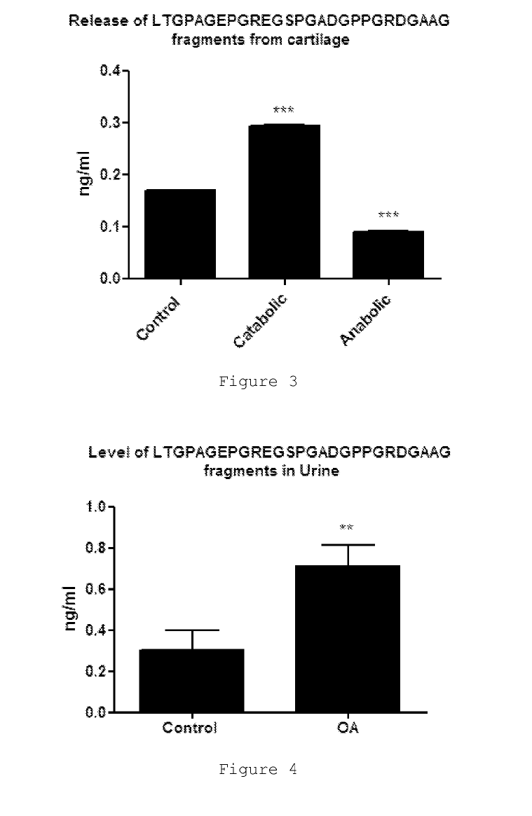

The native reactivity of the assay was tested by measurement of the release of the fragment from human articular cartilage cultured in catabolic and anabolic conditions. FIG. 3 shows that catabolic condition increases the release of fragments from the cartilage whereas the anabolic condition decreases the release of the fragments. This indicates that this fragment is a marker of cartilage turnover.

Measurement of 20 human urine samples originating from patients with OA and 20 samples from healthy subjects in the ELISA described above demonstrated an increase of >50% (p<0.05) in the concentration of the collagen type II fragments in the diseased subjects (FIG. 4).

### EXAMPLE 3

### Development of Immunoassay for Detection of Collagen Fragments Carrying One Neo-Epitopes—Test of the Whether this is a Genuine MMP Cleavage Site

One monoclonal antibody binding to the N-terminus of the amino acid sequence LTGPAGGGGC SEQ ID NO:124 as described above was peroxidase labelled according to standard procedures and used a biotinylated coater peptide in a competitive ELISA.

The coater peptide (LTGPAGGGGC-biotin SEQ ID NO:124) and antibody were pre-diluted in a 10 mM phosphate buffered solution (PBS) with bovine serum albumin (1%) and Tween 20 (0.1%) in a total volume of 100 μL and subsequently incubated in the microtitre plate with 20 μL of human serum sample. A synthetic peptide with the amino acid sequence LTGPAGEPGREGSPGADGPPGRDGAAG SEQ ID NO:62 containing both the N-terminal and C-terminal neo-epitope was used as calibrator. After two hours of incubation the wells were washed and incubated with a chromogen (TMB) for 15 minutes. Subsequently, the colour reaction was stopped by addition of 0.18M sulfuric acid.

To test whether this was actually MMP-derived, different inhibitors were added to cartilage explant cultures and the release of the fragment (LTGPAGGGGC SEQ ID NO:124) to the media was measured. FIG. 5 shows that the effect on the level when adding the protease inhibitors. The inhibitors used were following: metalloproteinase inhibitor (GM6001), cysteine protease inhibitor (E64), Aspartyl peptidase inhibitor Pepstatin A (Pep. A) and Serine protease inhibitor Aprotenin (apro.). Release was stimulated by adding catabolic cytokines to all conditions. Only GM6001 could block the release of the fragments, indicating that this a true MMP-derived fragment whilst the serine protease inhibitor, which indirectly inhibits MMPs, could also to a degree inhibit the release (FIG. 5). This confirms that the fragment is truly a MMP-derived fragment.

### EXAMPLE 4

### Development of Immunoassay for Detection of Collagen Fragments Carrying One Neo-Epitopes—Test of the Whether this is a Genuine MMP Cleavage Site

One monoclonal antibody binding to the C-terminus of the amino acid sequence GPPGRDGAAG SEQ ID NO:114 as described above was peroxidise labelled according to standard procedures and used a biotinylated coater peptide in a competitive ELISA.

The coater peptide (biotin-GPPGRDGAAG SEQ ID NO:114) and antibody were pre-diluted in a 10 mM phosphate buffered solution (PBS) with bovine serum albumin (1%) and Tween 20 (0.1%) in a total volume of 100 μL and subsequently incubated in the micro-titre plate with 20 μL of human serum sample. A synthetic peptide with the amino acid sequence LTGPAGEPGREGSPGADGPPGRDGAAG SEQ ID NO:62 containing both the N-terminal and C-terminal neo-epitope was used as calibrator. After two hours of incubation the wells were washed and incubated with a chromogen (TMB) for 15 minutes. Subsequently, the colour reaction was stopped by addition of 0.18M sulfuric acid.

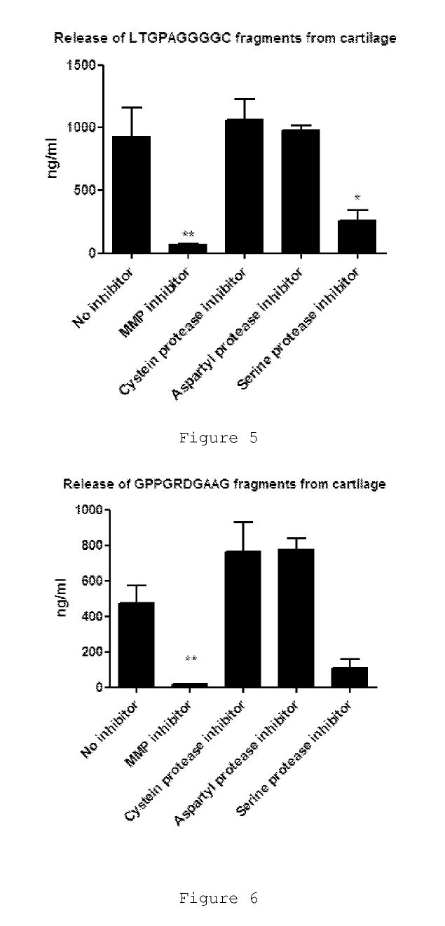

To test whether this fragment was actually MMP-derived, different inhibitors were add to cartilage explants cultures and the release of the fragment (GPPGRDGAAG SEQ ID NO:114) to the media was measured. FIG. 6 shows that the effect on the level when adding the protease inhibitors. The inhibitors used were following: metalloproteinase inhibitor (GM6001), cysteine protease inhibitor (E64), Aspartyl peptidase inhibitor Pepstatin A (Pep. A) and Serine protease inhibitor Aprotenin (apro.). Release was stimulated by adding catabolic cytokines to all conditions. Only GM6001 could block the release of the fragments, indicating that this a true MMP-derived fragment. The serine protease inhibitor, which indirectly inhibits MMPs, could also to some extent inhibit the release, although not significantly (FIG. 6). This confirms that the fragment is truly a MMP-derived fragment.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

Zhen E Y, Brittain I J, Laska D A, Mitchell P G, Sumer E U, Karsdal M A, Duffin K L. Characterization of metalloprotease cleavage products of human articular cartilage. Arthritis Rheum. 2008 August; 58(8):2420-31
Kafienah W, Brömme D, Buttle D J, Croucher L J, Hollander A P. Human cathepsin K cleaves native type I and II collagens at the N-terminal end of the triple helix. Biochem J. 1998 May 1; 331 (Pt 3):727-32
Dejica V M, Mort J S, Layerty S, Percival M D, Antoniou J, Zukor D J, Poole A R. Cleavage of type II collagen by cathepsin K in human osteoarthritic cartilage. Am J. Pathol. 2008 July; 173(1):161-9. Epub 2008 May 29
Dean, D. D., Martel-Pelletier, J., Pelletier, J. P., Howell, D. S., and Woessner, J. F., Jr. 1989. Evidence for metalloproteinase and metalloproteinase inhibitor imbalance in human osteoarthritic cartilage. J Clin Invest 84:678-685.
Reboul, P., Pelletier, J. P., Tardif, G., Cloutier, J. M., and Martel-Pelletier, J. 1996. The new collagenase, collagenase-3, is expressed and synthesized by human chondrocytes but not by synoviocytes. A role in osteoarthritis. J Clin Invest 97:2011-2019.
Hui, W., Rowan, A. D., Richards, C. D., and Cawston, T. E. 2003. Oncostatin M in combination with tumor necrosis factor alpha induces cartilage damage and matrix metalloproteinase expression in vitro and in vivo. Arthritis Rheum 48:3404-3418.
Schaller, S., Henriksen, K., Hoegh-Andersen, P., Sondergaard, B. C., Sumer, E. U., Tanko, L. B., Qvist, P., and Karsdal, M. A. 2005. In vitro, ex vivo, and in vivo methodological approaches for studying therapeutic targets of osteoporosis and degenerative joint diseases: how biomarkers can assist? Assay. Drug Dev. Technol. 3:553-580.
Sumer, E. U., Schaller, S., Sondergaard, B. C., Tanko, L. B., and Qvist, P. 2006. Application of biomarkers in the clinical development of new drugs for chondroprotection in destructive joint diseases: a review. Biomarkers 11:485-506.
Christgau, S., Garnero, P., Fledelius, C., Moniz, C., Ensig, M., Gineyts, E., Rosenquist, C., and Qvist, P. 2001. Collagen type II C-telopeptide fragments as an index of cartilage degradation. Bone 29:209-215.
Mouritzen, U., Christgau, S., Lehmann, H. J., Tanko, L. B., and Christiansen, C. 2003. Cartilage turnover assessed with a newly developed assay measuring collagen type II degradation products: influence of age, sex, menopause, hormone replacement therapy, and body mass index. Ann. Rheum Dis 62:332-336.
Reijman, M., Hazes, J. M., Bierma-Zeinstra, S. M., Koes, B. W., Christgau, S., Christiansen, C., Uitterlinden, A. G., and Pols, H. A. 2004. A new marker for osteoarthritis: cross-sectional and longitudinal approach. Arthritis Rheum 50:2471-2478.
Woolf A D, Pfleger B: Burden of major musculoskeletal conditions. Bull World Health Organ 2003; 81:646-56
Lawrence R C, Helmick C G, Arnett F C et al. Estimates of the prevalence of arthritis and selected musculoskeletal disorders in the United States. Arthritis Rheum 1998; 41:778-99.
Pritzker K P, Gay S, Jimenez S A, Ostergaard K, Pelletier J P, Revell P A, Salter D, van den Berg W B. Osteoarthritis cartilage histopathology: grading and staging. Osteoarthritis Cartilage 2006; 14:13-29.
Birmingham J D, Vilim V, Kraus V B. Collagen biomarkers for arthritis applications. Biomarker Insights 2006; 2: 61-76.
Fraser A, Fearon U, Billinghurst R C, Ionescu M, Reece R, Barwick T, Emery P, Poole A R, Veale D J. Turnover of type II collagen and aggrecan in cartilage matrix at the onset of inflammatory arthritis in humans: relationship to mediators of systemic and local inflammation. Arthritis Rheum. 2003 November; 48(11):3085-95.
Billinghurst R C, Dahlberg L, Ionescu M, Reiner A, Bourne R, Rorabeck C, Mitchell P, Hambor J, Diekmann O, Tschesche H, Chen J, Van Wart H, Poole A R.
Enhanced cleavage of type II collagen by collagenases in osteoarthritic articular cartilage. J Clin Invest. 1997 Apr. 1; 99(7):1534-45.
Poole A R, Ionescu M, Fitzcharles M A, Billinghurst R C. The assessment of cartilage degradation in vivo: development of an immunoassay for the measurement in body fluids of type II collagen cleaved by collagenases. J Immunol Methods. 2004 November; 294(1-2):145-53.
Otterness I G, Downs J T, Lane C, Bliven M L, Stukenbrok H, Scampoli D N, Milici A J, Mézes P S. Detection of collagenase-induced damage of collagen by 9A4, a monoclonal C-terminal neoepitope antibody. Matrix Biol. 1999 August; 18(4):331-41.
Downs J T, Lane C L, Nestor N B, McLellan T J, Kelly M A, Karam G A, Mezes P S, Pelletier J P, Otterness I G. Analysis of collagenase-cleavage of type II collagen using a neoepitope ELISA. J Immunol Methods. 2001 Jan. 1; 247(1-2):25-34.

1. A method of assay, comprising measuring in a biological sample fragments of a protein that contain an N-terminal neo-epitope and a C-terminal neo-epitope, each said neo-epitope being an amino acid sequence generated by protease cleavage of said protein, said method comprising binding the N-terminal neo-epitope with a first immunological binding partner specific for the presence of said N-terminal neo-epitope and binding the C-terminal neo-epitope with a second immunological binding partner specific for the presence of said C-terminal neo-epitope, and detecting the extent of dual binding of said binding partners.

2. A method as claimed in claim 1, wherein said assay is performed as a sandwich assay in which one of said immunological binding partners is immobilised to a solid support, said fragments are bound to said immobilised antibody and the binding of the other of said immunological binding partners to said fragments is detected.

3. A method as claimed in claim 1, wherein the assay is performed as a homogeneous sandwich assay.

4. A method as claimed in claim 1, wherein neither said first nor said second immunological binding partner specifically binds the intact protein from which said fragments derive.

5. A method as claimed in claim 4, wherein neither said first nor said second immunological binding partner specifically binds fragments of said protein containing the amino acid sequence of its respective said neo-epitope extended beyond the protease cleavage site.

6. A method as claimed in claim 1, wherein said neo-epitopes are from collagen type II.

7. A method as claimed in claim 1, wherein said neo-epitopes are produced by cleavage of collagen type II by an MMP or by cathepsin K.

8. A method as claimed in claim 7, wherein the first immunological binding partner is specific for an epitope defined by one of the following collagen type II cleavage amino acid sequences:

| | |
|---|---|
| [symbol] DQGVPG . . .; | SEQ ID NO: 54 |
| [symbol] REGSPG . . .; | SEQ ID NO: 55 |
| *LAGPKG . . .; | SEQ ID NO: 56 |
| and | |
| * [symbol] LTGPAG . . .. | SEQ ID NO: 57 |

9. A method as claimed in claim 7, wherein the second immunological binding partner is specific for an epitope defined by one of the following collagen type II cleavage amino acid sequences:

| | |
|---|---|
| . . . PKGARG [symbol] ; | SEQ ID NO: 58 |
| . . . GQPGPA [symbol] ; | SEQ ID NO: 59 |
| . . . EPGGVG [symbol] ; | SEQ ID NO: 60 |
| and | |
| . . . RDGAAG [symbol] . | SEQ ID NO: 61 |

10. A method as claimed in claim 1, wherein said first and second immunological binding partners are in combination specifically reactive with a collagen type II peptide fragment of the sequence:

| | |
|---|---|
| LTGPAGEPGREGSPGADGPPGRDGAAG | SEQ ID NO: 62 |
| or | |
| REGSPGADGPPGRDGAAG | SEQ ID NO: 63 |

11. A method as claimed in claim 7, wherein said immunological binding partner does not specifically bind a sequence as defined in claim 7 if continued past the indicated cleavage site.

12. An immunological assay kit comprising a first immunological binding partner specific for an epitope containing an isomerised amino acid residue and a second immunological binding partner specific for a protease generated neo-epitope.

13. A kit as claimed in claim 12 wherein said kit further comprises at least one of calibration standards immunoreactive with said binding partners, a wash reagent, a buffer, a secondary immunological binding partner for revealing binding between said first or second immunological binding partner and components of a sample, an enzyme label, an enzyme label substrate, a stopping reagent, and instructions for conducting an assay using said kit.
